(19) United States
(12) Patent Application Publication
(10) Pub. No.: US 2004/0166181 A1
(43) Pub. Date: Aug. 26, 2004

(54) Compositions, methods, and kits for weight loss and inhibiting the loss of lean body mass

(75) Inventors: John C. Hegenauer, Danville, CA (US); Haruna Yamaguchi, Castro Valley, CA (US); Winnie Wai-Yi Chan, Alamo, CA (US); Elaine L. Bagwell, Dublin, CA (US); Cindy Latham, Moraga, CA (US); Jeffrey M. Avila, Miami Beach, FL (US)

Correspondence Address: KLARQUIST SPARKMAN, LLP, 121 SW SALMON STREET, SUITE 1600, PORTLAND, OR, 97204, US

(73) Assignee: Shaklee Corporation

(21) Appl. No.: 10/722,368

(22) Filed: Nov. 24, 2003

Related U.S. Application Data
(60) Provisional application No. 60/428,993, filed on Nov. 22, 2002.

(51) Int. Cl. A61K 35/78; A61K 31/555

(52) U.S. Cl. 424/757; 514/184

(57) ABSTRACT

Disclosed are combinations of soy protein and chromium in amounts effective to inhibit the loss of lean body mass of a subject under caloric restriction. Further combinations include corosolic acid and optionally additional chromium. Still further combinations include a dietary composition of Fucus vesiculosus, Gambogia garcinia, Apis mellifica, Badiaga, Calcarea carbonica, Passiflora incarnata, Baryta carbonica, Calcarea fluorica, Lycopodium clavatum, Berberis vulgaris, Leptandra virginica, Thuja occidentalis, Galium aparine, Urtica urens, Histaminum muriaticum, and Sabadilla. Compositions of the disclosed soy protein, chromium, and corosolic acid combinations are disclosed as are kits containing the disclosed combinations as well as methods of using the disclosed combinations and compositions for weight loss and the inhibition of the loss of lean body mass in a subject during weight loss.

### PRIORITY

This disclosure claims priority to U.S. Provisional Application 60/428,993, filed Nov. 22, 2002, which is incorporated by reference herein.

### FIELD

The compositions, methods, and kits described herein relate to diets and dietary and nutritional supplements.

### BACKGROUND

An estimated 50% of individuals in the United States are overweight. Moreover, an estimated 50% of these individuals are sufficiently overweight to be considered obese. Obesity has been recognized as a public health problem in the United States, as well as the rest of the world.

Overweight or obese individuals are at higher risk for developing diseases such as hypertension, dyslipidemia, type-2 diabetes (non-insulin dependent diabetes mellitus or NIDDM), coronary heart disease, stroke, gallbladder diseases, osteoarthritis, sleep apnea, and respiratory problems. Such individuals also exhibit a higher prevalence of endometrial, breast, prostrate, and colon cancers. Further, higher than ideal body-weight is associated with an increase in all-causes of mortality.

The pharmaceutical industry is working diligently to develop drugs to help people lose weight. However, no drug has been discovered that allows individuals to eat all they desire and retain a sedentary lifestyle while simultaneously losing weight. Furthermore, the drug products available to the general public, whether by prescription or as over-the-counter preparations, are not free of risk. These risks include valvular heart disease arising out of the use of the combination of fenfluramine and phentermine (Fen-Phen), and irregular heart beat (arrhythmia) that is associated with the use of phenylpropanolamine (PPA). These risks resulted in a ban on the use of these drugs in weight loss products and programs.

Such risks are not limited to prescription and/or over-the-counter medications. The use of ephedra in nutritional products employed for weight loss has been associated with numerous incidences of arrhythmia in susceptible individuals taking such preparations.

Weight gain is a result of consuming more calories than are required by the body for its basal metabolic functions and the activities in which an individual is involved. The human body stores these excess calories as fatty deposits (lipids) throughout the body. Once stored, the body does not readily access these fatty deposits for energy. In order to employ these stored lipids as an energy source, the number of calories ingested must be less than the total energy expenditure of the body (basal metabolic rate plus activity level). In this way, the body makes up for the energy deficit by consuming fat as a source of fuel. However, the switch from ingested foods to the consumption of fat is not instantaneous. The body has feedback mechanisms that are directed toward preserving existing lipid stores. Therefore, in the interim between the initial reduction in caloric intake and the conversion of lipids to energy, the body consumes lean body mass as a source of energy. Hence, the body will consume some muscle tissue as its energy source during this period of conversion.

The use of lean body mass as an energy source is vitally important. Under conditions of caloric restriction muscle tissue represents the primary energy source to maintain the body's basal metabolic rate. It is also the primary tissue responsible for the consumption of fat once it is mobilized as an energy source. Any reduction in lean body mass represents a loss of tissue that can aid in the reduction of fat deposits in the body.

In order to lose weight, an individual must consume fewer calories than are expended. During this process individuals often have cravings for food. From a psychological standpoint, the ability to retain a reduction in ingested calories for a period of time sufficient to obtain any significant loss of weight is complicated by such cravings.

Although many food cravings are related to psychological sources, this is not their only source. Unless an individual is on a strictly protein and/or fat containing diet, the food ingested as part of their diet contains some measurable amount of carbohydrates. Once a carbohydrate is consumed and digested, it is absorbed into the bloodstream from the digestive tract. In response to an increase in blood glucose, the pancreas releases insulin. The purpose of insulin is to aid in the transport of glucose into the cells of the body where the glucose is employed as an energy source. However, if the amount of insulin released is greater than the amount of glucose present (which is often the case in overweight individuals), then the body reacts by signaling the brain to ingest more carbohydrates in order to balance the amount of insulin in the bloodstream. This insulin-induced craving for carbohydrates is very common during periods of caloric restriction.

Cravings for foods also can be traced to a lack of specific types of foods. For instance, individuals who attempt to lose weight by eating a high protein, low fat diet often find themselves craving food that contain fats. Although there are many “fat-free” foods available in commercially available food, these products often lack the palatability provided by the presence of fat.

The use of soy protein in combination with soybean fibers, and optionally also in combination with other vitamins and minerals (such as iron, zinc, iodine, manganese, chromium, and selenium) has been described in U.S. Pat. No. 6,268,011 as a food supplement or for lowering lipids in serum.

The use of a chromium salt in nutritional products has been the subject of many patents, for instance, U.S. Pat. Nos. 4,954,492; 5,087,623; 5,175,156; 5,194,615; 6,251,888; 6,251,889; 6,323,192; 6,432,942; and 6,471,998. These patents describe the use of a chromium salt, either alone or in combination with other ingredients, in lowering blood levels of lipids and/or controlling blood glucose levels. U.S. Pat. Nos. 5,087,624; 6,251,889; 6,323,192; 6,432,942; and 6,471,998 describe the use of chromium salts either alone or in combination with other materials for increasing lean body mass. In this latter context, the chromium salt is employed as an anabolic agent to increase muscle mass when taken in combination with a strength building exercise program. U.S. Pat. Nos. 6,277,842; 6,399,089; and 6,413,545 also describe the use of a chromium salt in dietary preparations.

### SUMMARY

It has been discovered that consuming a combination of soy protein and chromium while following a restricted calorie diet inhibits the loss of lean body mass.

Disclosed herein are compositions for inhibiting the loss of lean body mass under conditions of caloric restriction. These compositions include soy protein and chromium in amounts such that the composition is effective to inhibit the loss of lean body mass in an individual under caloric restriction. For example, certain embodiments of such compositions contain at least about 8 grams (g) of soy protein and at least about 100 micrograms (μg) of chromium. In some cases the compositions are in the form of a dry drink powder. In other cases the compositions are in the form of a shake drink.

Also disclosed are weight loss methods that include consuming (or providing instructions for consuming) a combination of soy protein and chromium under conditions of caloric restriction in amounts sufficient to inhibit the loss of lean body mass. The combination can be consumed concurrently (for example in a composition including soy protein and chromium) or non-concurrently (for example by consuming the soy protein and chromium separately but within a sufficient period of time to have the weight loss effect). In some instances, the method also includes following a calorically restricted diet. Further disclosed is a method of treating overweight subjects by selecting a subject that is overweight and administering to the subject (for example by instructing the subject to consume) a combination of soy protein and chromium in amounts effective to inhibit the subject's loss of lean muscle mass under conditions of caloric restriction. The combination can be administered or consumed concurrently or non-concurrently as described above. In some instances, the subject follows or is instructed to follow a calorically restricted diet.

Kits are also provided for inhibiting the loss of lean body mass under conditions of caloric restriction. The kit includes soy protein, chromium and, in some cases, also includes instructions for consuming amounts of the soy protein and the chromium effective to inhibit the loss of lean body mass under conditions of caloric restriction. In certain cases instructions for a calorically restricted diet are also included.

Further, it has been discovered that combining the above disclosed soy protein and chromium combination with corosolic acid, and optionally additional chromium, leads to weight loss and the retention of lean body mass during weight loss in subjects. This is the case even in the absence of regimented exercise or conscious caloric restriction. Accordingly, in some embodiments, the disclosed soy protein and chromium combination and/or composition further includes corosolic acid and additional chromium. The corosolic acid and additional chromium in some embodiments are provided in a separate nutritional supplement. Also, the above disclosed methods of losing weight in some embodiments include administering (for example by providing instructions to consume) corosolic acid and optionally chromium. Methods of losing weight with the soy protein/chromium combination and corosolic acid and optionally additional chromium are disclosed that do not require conscious caloric restriction, yet still result in weight loss and the retention of lean body mass. Kits containing the soy protein/chromium combination and corosolic acid and optionally additional chromium also are disclosed.

In addition, it has been discovered that combining the soy protein and chromium combination with the corosolic acid, and optionally additional chromium, and with a dietary composition that comprises an effective amount of Fucus vesiculosus, Gambogia garcinia, Apis mellifica, Badiaga, Calcarea carbonica, Passiflora incarnata, Baryta carbonica, Calcarea fluorica, Lycopodium clavatum, Berberis vulgaris, Leptandra virginica, Thuja occidentalis, Galium aparine, Urtica urens, Histaminum muriaticum, and Sabadilla, in addition to inhibiting the loss of lean body mass during weight loss under conditions of caloric restriction, also leads to weight loss in subjects without conscious caloric restriction. Accordingly, the above disclosed methods of losing weight in some embodiments include administering (for example by providing instructions to consume) the disclosed dietary composition. Methods of losing weight with the soy protein/chromium combination and corosolic acid and the disclosed dietary composition also are disclosed that do not require conscious caloric restriction. In addition, kits containing the soy protein/chromium combination, corosolic acid, and optionally additional chromium, and the disclosed dietary composition also are disclosed.

Further, methods are disclosed of causing weight loss in subjects having Metabolic Syndrome by administering the above disclosed compositions and combinations. In certain embodiments of the method, the compositions and combinations are provided to the subject, who is instructed in their use.

### DETAILED DESCRIPTION

As used herein, the terms individual or subject refer to an animal, such as a mammal, for example a human.

“Metabolic Syndrome” is a disease indicated by a concurrence of disturbed glucose and insulin metabolism, overweight and abdominal fat distribution, mild dyslipidemia, and hypertension, and is associated with subsequent development of type 2 diabetes mellitus and cardiovascular disease (CVD). As used herein, a subject having Metabolic Syndrome has at least three of the following clinical features: a waist circumference greater than about 102 cm in men and about 88 cm in women; serum triglycerides level of at least about 150 mg/dL (1.69 mmol/L); high-density lipoprotein cholesterol level of less than about 40 mg/dL (1.04 mmol/L) in men and about 50 mg/dL (1.29 mmol/L) in women; blood pressure of at least about 130/85 mm Hg; or serum glucose level of at least about 110 mg/dL (6.1 mmol/L). This definition corresponds to the recent definition noted in the Journal of the American Medical Association. Ford et al, JAMA, 287:356-359 (2002).

Waist circumference is measured around the subject's waist along a substantially horizontal line at a point just below the subject's naval, for example with a measuring tape. The other above noted criteria are evaluated by any reliable means, such as those conventionally used in medical examinations. Serum triglycerides, glucose levels and high-density lipoprotein cholesterol levels are measured, for example by standard blood chemistry panels. Blood pressure is measured for example by sphygmomanometry.

Corosolic acid (2-alpha-3-beta-dihydroxy-12-ursen-28-oic acid; 3-alpha, 3-beta-di-hydroxyursolic acid) refers to a triterpene compound that can be extracted from the leaves of the plant Lagerstroemia speciosa (banaba leaf) and Punica granatum and also is known as colosolic acid and botanical insulin. As used herein corosolic acid also refers to equivalent effective amounts of pharmaceutically acceptable salts, analogs, derivatives, isomers, and metabolites of corosolic acid such as, methyl and glucoprano-syl esters (at the carbonyl group), and methyl, acetyl, or cinnamoyl substitutions (at one or more hydroxyl group) that retain the desired biological effect of corosolic acid.

Certain embodiments of compositions disclosed herein are described as dilutions of certain substances. Dilutions prepared and designated by the suffix “X” are diluted about one part of substance to about nine of a liquid solution, such as alcohol and water. Those designated by the suffix “C” are diluted about one part of substance to about 99 parts liquid. The number preceding the ‘X’ or ‘C’ is the number of times the one in ten or one in a hundred dilution takes place. “Succussion” is the process whereby each successive dilution is vigorously shaken, for example, between ten and over one hundred times, in order to mix the substance with the liquid. Thus 3X is a substance diluted one part in ten, three successive times and “succussed” in between each dilution. A 30C preparation is one that has been diluted one in a hundred, 30 times and succussed in between each dilution. An individual's lean body mass is the mass of the individual that is muscle and connective tissue. Under conditions of caloric restriction an individual loses body weight in the form of both lean body mass and fat. Loss of lean body mass is “inhibited” when, under conditions of caloric restriction, the relative loss of lean body mass is less than would be expected based on the difference between the number of calories ingested and expended.

Soy protein/chromium compositions for inhibiting the loss of the lean body mass under conditions of caloric restriction are disclosed herein. Such compositions include soy protein and chromium in amounts such that the composition is effective to inhibit the loss of lean body mass in an individual under conditions of caloric restriction.

Soy protein refers to protein obtained from the soybean plant. In some cases the soy protein is isolated from the soybean by methods well known in the art. In some instances the isolated soy protein is prepared from cleaned, dehulled soybeans by removing a majority of the non-protein components so that the isolated product contains at least about 90% protein by weight. The preparation takes place through a series of steps in which the soybean protein portion is separated from the rest of the soybean. In particular examples, bland tasting soy protein materials are used. Further, although genetically modified soy (GM soy) protein is used in some cases, non-genetically modified soy (Non-GM soy) protein is also used. Such soy proteins are available from a variety of companies such as Protein Technologies, Inc. An effective amount of soy protein for use in inhibiting the loss of lean body mass is at least about 12 g per serving or dose. In particular examples an effective amount of soy protein is from about 12 g to about 50 g per serving or dose. In certain examples an effective amount of soy protein is from about 12 g to about 14 g per serving or dose. In a particular example an effective amount of soy protein was from about 13 g to about 14 g per serving or dose.

As used herein, chromium refers to chromium in a biologically acceptable salt or chelate of chromium. It is provided in any bioactive and physiologically acceptable form. Though the chromium is provided in many forms, when referring to an amount of chromium herein it is meant the amount of actual chromium in the biologically acceptable salt or chelate (for example, 180 μg of chromium is provided by about 1.4 mg of the biologically acceptable chelate chromium nicotinate). In some cases the chromium is in the form of a chromium salt, such as chromium chloride, while in other cases it is in the form of a chromium chelate, such as chromium nicotinate (including mononicotinate, dinicotinate, trinicotinate, and polynicotinate), chromium picolinate (including monopicolinate, dipicolinate, and tripicolinate), protein chelates, and chelates of any bioavailable organic acid (such as amino acids), or compositions or combinations thereof, such as a composition of chromium mononicotinate, dinicotinate, trinicotinate, and polynicotinate or monopicolinate, dipicolinate, and tripicolinate. In particular examples chromium is provided in the form of chromium nicotinate or picolinate. An effective amount of chromium for use in inhibiting the loss of lean body mass is from at least about 100 μg per serving or dose and ranges to about 1 milligram (mg) per serving or dose. In particular examples an effective amount of chromium is from about 120 μg to about 800 μg per serving or dose. In one particular example about 180 μg of chromium per serving or dose was an effective amount. The different forms in which chromium is provided contain varying amounts of chromium. One of ordinary skill in the art would be able to determine the amount of a particular form of chromium necessary to provide a particular amount of chromium. In one particular example, an effective amount of chromium was about 180 μg of chromium per serving or dose and was provided in the form of about 1.4 milligrams (mg) of chromium nicotinate. In particular examples, the ratio of chromium to soy protein is at least about 10. In other cases the ratio is at least about 12, for example from about 12 to about 30.

As used herein, a condition of caloric restriction refers to following a calorically restricted diet under which an individual on average consumes fewer calories than the individual expends in a relevant period, such as a day, a week, a month, or longer. In some cases an individual follows a diet under which the individual consumes less than about 2,000 calories per day (on average), for example, a diet of from about 1,000 to about 1,500 calories per day, or in other cases a diet of from about 1,200 to about 1,400 calories per day. In still other cases an individual follows a diet of less than 1,200 calories per day. In some examples, an individual remains under conditions of caloric restriction for from about a day to about a year or longer, for example, for about one week, for about one month, for about 8 weeks, for about 12 weeks, or for about one year.

In some cases the soy/chromium composition includes other ingredients for added nutrition, preservation, or flavor, such as fructose, high oleic sunflower oil powder, acacia gum, canola oil, inulin, milk protein isolate, dicalcium phosphate, silicon dioxide, sodium citrate, potassium chloride, lecithin, whey protein isolate, guar gum, flavoring, ferrous fumarate, sweeteners (for example sucralose) mixed tocopherol concentrate (including d-alpha tocopheryl acetate), and vitamin and mineral premixes. Vitamin premixes include, for example, premixes of sodium citrate, ascorbic acid, yeast, vitamin A palmitate, vitamin B 12, vitamin D3, pyridoxine hydrochloride, riboflavin, thiamine mononitrate, niacinamide, folic acid, and biotin. Mineral premixes include, for example, potassium chloride, magnesium oxide, molybdenum, compounds selenium yeast, zinc oxide, copper gluconate, calcium pantothenate, manganese sulfate, and potassium iodide. In certain specific cases where these other ingredients are included in a soy protein and chromium composition, the weight of the soy protein is from about 10% to about 50% of the total weight of the composition, for example, the soy protein is from 20% to about 40% of the total weight of the composition or, in an even more specific example, from about 27% to about 30% of the total weight of the composition, and the weight of the chromium in the bioactive form provided, such as chromium nicotinate, is from about 0.0005% to about 0.10%, of the total weight of the composition, for example, the weight of the chromium is from 0.002% to about 0.01% of the total weight of the composition or, in an even more specific example, from about 0.003% to about 0.004% of the total weight of the composition. In certain instances the other ingredients are also used in certain relative amounts. In certain cases the weight of fructose is about 20% to about 30% of the total weight of the composition, for example the weight of fructose is about 23% to about 28% of the total weight of the composition, the weight of high oleic sunflower oil powder is about 8% to about 18% of the total weight of the composition, for example the weight of high oleic sunflower oil powder is about 11% to about 13% of the total weight of the composition, the weight of acacia gum is about 10% to about 15% of the total weight of the composition, for example, the weight of acacia gum is about 14% to about 16% of the total weight of the composition, the weight of canola oil is about 1% to about 5% of the total weight of the composition, for example, the weight of canola oil is about 2% to about 3% of the total weight of the composition, the weight of inulin is about 1% to about 5% of the total weight of the composition, for example, the weight of inulin is about 2% to about 3% of the total weight of the composition, the weight of milk protein isolate is about 0.5% to about 5% of the total weight of the composition, for example, the weight of milk protein isolate is about 1% to about 2% of the total weight of the composition, the weight of dicalcium phosphate is about 0.5% to about 3% of the total weight of the composition, for example, the weight of dicalcium phosphate is about 0.5% to about 3% of the total weight of the composition, the weight of silicon dioxide is about 1% to about 2% of the total weight of the composition, for example, the weight of silicon dioxide is about 1.0% to about 1.75% of the total weight of the composition, the weight of sodium citrate is about 0.5% to about 3% of the total weight of the composition, for example, the weight of sodium citrate is about 1% to about 2% of the total weight of the composition, the weight of potassium chloride is about 0.5% to about 3% of the total weight of the composition, for example, the weight of potassium chloride is about 1% to about 2% of the total weight of the composition, the weight of lecithin (such as soy lecithin) is about 0.5% to about 3% of the total weight of the composition, for example, the weight of lecithin (such as soy lecithin) is about 1% to about 1.5% of the total weight of the composition, the weight of whey protein isolate is about 0.05% to about 2% of the total weight of the composition, for example, the weight of whey protein isolate is about 0.9% to about 0.95% of the total weight of the composition, the weight of guar gum is about 0.1% to about 2% of the total weight of the composition, for example, the weight of guar gum is about 1.5% to about 2% of the total weight of the composition, the weight of flavoring is about 0.0% to about 2% of the total weight of the composition about 1% to about 2% of the total weight of the composition, for example, the weight of flavoring is about 0.7% to about 1.1% of the total weight of the composition, the weight of vitamin premix is about 0.05% to about 2% of the total weight of the composition, for example, the weight of vitamin premix is about 0.4% to about 0.5% of the total weight of the composition, the weight of mineral premix is about 0.05% to about 2% of the total weight of the composition, the weight of mineral premix is about 0.3% to about 0.4% of the total weight of the composition, the weight of ferrous fumarate is about 0.005% to about 0.05% of the total weight of the composition, for example, the weight of ferrous fumarate is about 0.02% to about 0.03% of the total weight of the composition, the weight of the sweetener (such as sucralose) is about 0.00% to about 0.10% of the total weight of the composition, for example, the weight of the sweetener is about 0.001% to about 0.01% or about 0.008 to about 0.009% of the total weight of the composition, and the weight of mixed tocopherol is about 0.0005% to about 0.005% of the total weight of the composition, for example, the weight of mixed tocopherol is about 0.001% to about 0.003% of the total weight of the composition. Other additional nutritional supplements, preservatives, and flavorings are also included in some instances. One of ordinary skill in the art would be able to determine appropriate combinations of these ingredients and others for adding nutrition to the composition, preserving the composition, and/or adding flavor to the composition.

In some cases the soy/chromium composition is a dry drink powder. For example, each component of the composition is mixed together into a powder. In one specific case such a powder is made by combining the liquid ingredients (lecithin, canola oil and mixed tocopherol concentrate) in a vessel and mixing these ingredients to form a homogenous mixture. In a separate vessel all the dry ingredients including the soy protein and the chromium are combined (except silicon dioxide and a portion of high oleic sunflower oil powder). The vessel may be a blending device, or the dry ingredients can be transferred to an appropriate blending device, preferably one equipped with a chopping device. The dry ingredients are then mixed thoroughly.

While mixing the dry ingredients, the liquid ingredients are applied onto the well-blended dry ingredients in an even and consistent fashion, preferably utilizing a spraying device. After the addition of the liquid ingredients, the mixture is mixed (such as by tumbling) and chopped until the entire mixture is well blended. The remainder of the high oleic sunflower oil powder and silicon dioxide are then added to the mixture and the entire mixture is blended until these ingredients are also well blended into the mixture, which is now a dry powder.

In other cases the soy/chromium composition is a shake drink. For example, in some instances a dry drink powder is mixed with a liquid such as water (for example, purified water), milk, or juice and agitated to mix the liquid and the powder to create a shake drink. This mixing is sometimes performed before the composition is packaged, and sometimes is performed by the consumer of the composition. In still other cases the composition comprises a nutrition bar. In certain specific examples the relative amounts of the components of the shake drink, as expressed in weight of the ingredient to the total weight of the shake drink are from about 60% to about 95% liquid, such as purified water, for example from about 70% to 80% liquid, from about 2% to about 30% soy protein, for example from about 2% to about 15% soy protein, from about 0% to about 5% other proteins, such as whey protein or milk protein or combinations thereof, for example, from about 2% to about 4% other protein, from about 2% to about 12% carbohydrates, for example, from about 4% to about 8% carbohydrates, from about 0.5% to about 5% fats (lipids), for example, from about 2% to about 3% fats, from about 5% to about 15% fiber, for example, from about 8% to about 12% fiber, a trace percentage of chromium, for example from about 80 μg to about 800 μg, and vitamin premix, mineral premix, and other ingredients as desired.

The soy/chromium compositions can be made to have any amount of calories. In some examples, the soy/chromium compositions contain from about 100 to about 300 calories. In particular instances the compositions contain about 200 calories.

In some embodiments the soy/chromium composition further includes corosolic acid and, optionally, additional chromium (referred to herein as the soy protein/chromium/corosolic acid composition). For example in some embodiments the soy/chromium composition further includes about 0.05 to about 1 mg of corosolic acid, such as about 0.1-0.6 mg, about 0.15-0.40 mg, about 0.25-0.35 mg, or, for example, about 0.32 mg of corosolic acid. The corosolic acid in some cases is provided in the form of banaba leaf extract, such as banaba leaf extract standardized to contain about 1% corosolic acid, for example about 5 to about 100 mg of such a standardized banaba leaf extract, such as about 10-60 mg, about 15-40 mg, about 25-35 mg, or, for example, about 32 mg of banaba leaf extract. Optionally, the soy protein/chromium/corosolic acid composition includes 100-600 μg of additional chromium, such as about 200-500 μg, about 300-400 μg, such as about 400 μg of chromium. In still further embodiments of the soy protein/chromium/corosolic acid composition, the composition includes one or more of magnesium (such as magnesium oxide), zinc (such as zinc gluconate), taurine, vanadium (such as vanadium amino acid chelate), and alpha lipoic acid. Certain of such embodiments contain about 100-300 mg, such as about 150-250 mg, for example about 200 mg of magnesium; about 1-10 mg, such as about 3-7 mg, for example about 5 mg of zinc; about 300-700 mg, such as about 400-600 mg, for example about 500 mg of taurine; about 25-175 μg, such as about 50-150 μg, for example about 100 μg vanadium; and/or about 2.5-20 mg, such as about 5-15 mg, for example about 10 mg of alpha lipoic acid. Any of the further components of the soy protein/chromium composition noted in this paragraph can be mixed with embodiments of the soy protein/chromium compositions disclosed above.

Also disclosed are methods of losing weight that include consuming a combination of soy protein and chromium corresponding to the soy protein and chromium of the disclosed soy protein/chromium composition (the soy protein/chromium combination) under conditions of caloric restriction in amounts sufficient to inhibit the loss of lean body mass. In some cases the soy protein/chromium combination is consumed as a soy protein/chromium composition, as discussed above. In other cases the soy protein and the chromium are consumed separately. In some instances, the method includes instruction regarding a calorically restricted diet. In certain embodiments the method includes weight loss by a subject having Metabolic Syndrome.

Further disclosed are methods of treating an overweight subject by selecting a subject that is overweight and administering to the subject (for example, instructing or otherwise causing the subject to consume) the combination of soy protein and chromium in amounts effective to inhibit the subject's loss of lean muscle mass under conditions of caloric restriction. In some cases the combination is administered as the disclosed soy protein/chromium composition. In other cases the soy protein and the chromium are administered separately. In some instances, the subject is instructed regarding a calorically restricted diet and or following a calorically restricted diet. As used herein, a method of losing weight refers to a conscious effort to reduce body weight, whether measured in body weight or another measure of weight loss (for example, body mass index (BMI)).

Selecting an overweight subject includes selecting an individual with excess of body weight that puts the subject at risk for complications associated with being overweight. For example, weight-related complications include hypertension, dyslipidemia, type-2 diabetes (non-insulin dependent diabetes mellitus or NIDDM), coronary heart disease, stroke, gallbladder diseases, osteoarthritis, sleep apnea, and respiratory problems. Typically, being overweight refers to an excess of body weight compared to set standards. A generally accepted standard for determining whether a human is overweight is BMI. An individual's BMI is calculated as weight in kilograms (kg) divided by height in meters squared (m²). Generally an individual having a body mass index (BMI) of at least 25 is considered overweight. However, BMI is not always an accurate measurement of whether an individual is overweight for the purposes of determining whether the individual's weight constitutes a health risk. For example, a muscular athlete may have a high BMI because of the large amount of weight the individual carries as muscle, but not have the health risks generally associated with being overweight. Other indicators such as percentage body fat are also used in making this determination. Additionally, the location of an individual's body fat is also relevant. Individuals with body fat concentrated in the abdominal region and/or around the hips are at a higher risk for most overweight associated complications than individuals with body fat concentrated in other areas, such as the legs. One of ordinary skill in the art would be able to determine whether a person is overweight in this medically relevant sense.

An obese individual is a particular example of an overweight person. As used herein the term obese refers to an individual having an abnormally high proportion of body fat. Typically this is determined by measuring an individual's BMI. An individual with a BMI of 30 or higher would generally be considered obese. However, as mentioned above, BMI is not always a reliable measure for assessing the weight associated health risks for an individual. However, one of ordinary skill in the art would be able to determine whether an individual is obese in situations where BMI does not accurately determine the individual's obesity.

In certain cases selecting a subject that is overweight further includes selecting a subject having Metabolic Syndrome. Such selection, in some cases, includes testing the subject for the Metabolic Syndrome clinical features disclosed above.

In some cases the soy protein/chromium combination is consumed orally in any ingestible form. In some instances the soy protein/chromium combination is consumed as a soy protein/chromium composition including soy protein and chromium, as described above. In some instances the soy protein/chromium composition is consumed as a dry powder, in others as a shake drink, and in still others in bar form. In some examples the soy protein/chromium combination is consumed using enteral delivery methods, such as through a nasogastric tube or percutaneous endoscopic gastrostomy.

In still other cases the soy protein and the chromium of the soy protein/chromium combination are consumed separately, but within a sufficient period of time from one another to have the desired effect. In cases where the soy protein and chromium are consumed separately, the soy protein is consumed orally or enterally in any ingestible form. In some instances the soy protein is consumed in the form of soymilk. In other instances the soy protein is consumed in the form of a soy protein bar. In other instances the soy protein is consumed in a shake drink including soy protein powder and a liquid such as water, milk, or juice. In still other instances the soy protein is consumed in a shake drink composition including soy protein and other ingredients for added nutrition, preservation, or flavor, such as those discussed above that are in some cases added to the disclosed compositions. Other additional nutritive supplements, preservatives, and flavorings are also included in some instances.

In cases where the soy protein and chromium of the soy protein/chromium combination are consumed separately, the chromium is consumed orally or enterally in any ingestible form, such as capsules (hard or soft), tablets, elixirs, powders, granules, suspensions in water or non-aqueous media, or as an additive to food or beverages. In some cases the chromium is mixed with a pharmaceutical carrier (conventional tableting ingredients such as corn starch, lactose, maltodextrin, sucrose, sorbitol, talc, stearic acid, magnesium stearate, dicalcium phosphate or gums) and/or other pharmaceutical diluents, such as water, to form a solid preformulation composition containing a substantially homogenous mixture of the composition, or a non-toxic pharmaceutically acceptable salt thereof. When referring to the preformulation compositions as substantially homogenous, it is meant that the active ingredients are dispersed evenly throughout the composition so that the composition may readily be subdivided into equally effective unit dosage forms such as capsules, pills, and tablets. In other cases the chromium is consumed in liquid preparations for oral administration, such as solutions, syrups, or suspensions.

Such liquid preparations are prepared by conventional means with pharmaceutically acceptable additives such as suspending agents (sorbitol syrup, methyl cellulose, non-hydrogenated edible fats or hydrogenated edible fats), emulsifying agents (lecithin or acacia), non-aqueous vehicles (almond oil, oily esters, or ethyl alcohol), preservatives (methyl or ethyl p-hydroxybenzoates or sorbic acid), and artificial or natural colors and/or sweeteners.

The effective amounts of soy protein and chromium for use in the disclosed methods of losing weight under caloric restriction are the same as discussed above for use in the disclosed soy protein/chromium compositions. In some particular cases an individual consumes from about 8 g to about 50 g of soy protein per day, for example from about 12 g to about 14 g of soy protein per day, and consumes from about 100 μg to about 1 mg of chromium per day, for example, from about 100 μg to about 360 μg of chromium per day, or in some specific cases from about 120 μg to about 180 μg of chromium per day. In some examples these effective amounts are consumed in a single serving or dose of the disclosed composition or combinations. In other cases these effective amounts are consumed by ingesting multiple servings or doses per day, for example, two servings per day of a composition each containing half of the effective amount of soy protein chromium.

The disclosed methods include an individual or subject consuming and/or being administered the disclosed soy protein/chromium combinations or compositions for any length of time. In certain cases the soy protein/chromium combinations (including the disclosed compositions) are consumed for a week or more, for example for up to a year or more. In particular examples, the combinations are consumed for a period of from about 8 weeks to about 12 weeks, for example, either 8 weeks or 12 weeks. In other cases the combinations are consumed for a period of about five days, or more.

In some cases, an individual or subject is instructed regarding a calorically restricted diet. In some instances the subject is orally instructed concerning the need to consume fewer calories than the subject expends to achieve a condition of caloric restriction. In other instances instructions are provided in written form. In certain cases the subject is also advised regarding how to determine the caloric content of foods by reading the information labeling on food products or by accessing other data regarding the caloric content of various foods. In some instances, the subject is instructed concerning the effects of portion or serving sizes of various foods on weight loss. In some instances the subject is also instructed concerning obtaining adequate nutrition during periods of caloric restriction as well as warning signs of improper or dangerous caloric restriction and nutritional deficiencies.

In some cases the individual or subject is instructed regarding participation in exercise with either oral or written instructions to participate in exercise such as walking at least one mile or for at least 30 minutes each of at least five days a week, or exercise with a similar or higher level of exertion. Still other embodiments include performance of such exercise by the subject or individual.

In some embodiments the methods disclosed above further include consuming corosolic acid, and, optionally, additional chromium as well as one or more of magnesium, zinc, taurine, vanadium, and alpha lipoic acid. These further substances, in some embodiments, are additional components of the soy protein/chromium composition, as in the soy protein/chromium/corosolic acid composition disclosed above. In other embodiments these further substances are consumed separately from the soy protein/chromium composition, for example in a separate nutritional supplement or supplements. Such a nutritional supplement is made in some cases in accordance with the discussion above relating the consumption of chromium in cases where the soy protein and chromium of the soy protein/chromium composition are consumed separately, for example by mixing with a pharmaceutical carrier to form a tablet. In any case, the effective daily amounts of the corosolic acid and the other further components disclosed in this paragraph are the same as disclosed above for inclusion in the soy protein/chromium/corosolic acid composition. These effective amounts are consumed in a single serving or dose in some embodiments, and are consumed in multiple servings or doses in other embodiments. For example, in some embodiments where the separate nutritional supplement containing these further components is consumed, the effective daily amounts of the components of the nutritional supplement are divided into two or more doses, such as two or more tablets. However, in other embodiments, such as embodiments where the soy protein and chromium of the soy protein/chromium combination are consumed separately, the optional additional chromium disclosed in this paragraph is consumed as part of the chromium of the soy protein/chromium combination and the corosolic acid is consumed as part of the separate nutritional supplement.

In embodiments wherein the nutritional supplement disclosed in the paragraph above is consumed separately from the soy protein/chromium composition, the nutritional supplement is consumed at any time or times of the day. In certain embodiments the nutritional supplement is consumed at or near the time of one or more meals in a day, such as the largest meal of the day, for example within half an hour of the meal or during the meal. In some embodiments, the nutritional supplement is consumed in two or more doses, for example at or near the time of the two largest meals of the day, or at or near the time of each meal of the day.

In certain embodiments where the soy protein/chromium combination and the corosolic acid (optionally with the additional chromium and other further components) are consumed, subjects are not instructed regarding a calorically restricted diet are not required to follow any particular diet, and/or do not consciously follow a calorically restricted diet.

Still other embodiments of the disclosed methods include consuming the soy protein/chromium combination, the corosolic acid (and optionally the additional chromium and other components), and further include consuming (for example by oral consumption, or other mucosal administration, such as oral, nasal or sublingual administration) a dietary composition comprising Fucus vesiculosus (sea kelp), Gambogia garcinia (gummi gutti), Apis mellifica (honeybee), Badiaga (fresh water sponge), Calcarea carbonica carbonate of lime), Passiflora incarnata (passion flower), Baryta carbonica (carbonate of Baryta), Calcarea fluorica (fluoride of lime), Lycopodium clavatum (club moss), Berberis vulgaris (barberry), Leptandra virginica (culver's root), Thuja occidentalis (arbor vitae), Galium aparine (goose grass), Urtica urens (stinging nettle), Histaminum muriaticum (histamine hydrochloride), and Sabadilla (cevadilla seed) as active ingredients. In some cases the disclosed dietary composition includes dilutions of these substances in a liquid solution, such as a water and/or alcohol solution. These substances are diluted in some embodiments from about 3X to about 200C. In certain embodiments the dietary composition includes dilutions of these substances of about 1-6X Fucus vesiculosus, such as 3X; about 1-6X Gambogia garcinia, such as 3X; about 10-50C Apis mellifica, such as 30C; about 1-7X Badiaga, such as 4X; about 10-50X Calcarea carbonica, such as 30X; about 1-6X Passiflora incarnata, such as 3X; about 6-18X Baryta carbonica, such as 12X; about 6-18X Calcarea fluorica, such as 12X; about 2-10X Lycopodium clavatum, such as 6X; about 2-10X Berberis vulgaris, such as 6X; about 1-6X Leptandra virginica, such as 3X; about 2-10X Thuja occidentalis, such as 6X; about 1-6X Galium aparine, such as 3X; about 10-50C Urtica urens, such as 30C; about 100-300C Histaminum muriaticum, such as 200C; and about 10-50C Sabadilla, such as 30C. In some embodiments the dietary composition is provided as a dietary spray, and each dilution is included in an about equal amount as each other dilution. In certain embodiments the dilutions are further mixed with a carrier liquid, such as water or alcohol to form a solution of about 80% active dilutions and about 20% carrier by volume.

In some embodiments the disclosed dietary composition is taken by the subject prior to one or more meals each day, such as about 5-30 minutes prior to a meal, for example about 15 minutes. In certain embodiments the subject consumes the dietary composition before the subject's largest meal of the day, before the subject's two largest meals of the day, or before each meal of the day. The effective daily dose of the dietary composition is about 0.5-3 mL, such as about 1-2 mL, for example about 1 mL. Where the dietary composition is consumed more than once per day, the effective dose can be divided into the number of doses consumed per day. In some embodiments the disclosed dietary composition is administered orally by use of spray bottle (such as a pump-siphon spray bottle) that dispenses about 0.1-0.5 mL mL per spray. A subject consuming the effective dose by consuming the dietary composition before the subject's two largest meals of the day and administering the dietary composition with the spray bottle just described in some embodiments would consume two sprays from the spray bottle before each of the subject's two largest meals. In certain embodiments where spray administration is used, the spray is administered under the subject's tongue (sublingually).

In certain embodiments of the disclosed methods where the disclosed dietary composition is used, the dietary composition is administered (for example consumed) over the period of performance of the method in alternating periods during which the compositions are consumed or not consumed. For example, the dietary composition in some cases is consumed as disclosed for a period of time, such as one to three weeks, and then the subject refrains from consuming the dietary composition for a period of time, such as about one to three weeks, before again consuming the dietary composition. In certain embodiments the period of consumption is three weeks and the interval without consumption is one week. In other embodiments the dietary composition is consumed for the entire period the method is performed, such as for five days, 8 weeks, 12 weeks, or longer.

In certain embodiments of the method, subjects are not instructed regarding a calorically restricted diet, are not required to follow any particular diet, and/or do not consciously follow a calorically restricted diet. In other embodiments, subjects are not instructed regarding an exercise regimen.

Further disclosed are kits for inhibiting the loss of lean body mass under conditions of caloric restriction. These kits include soy protein and chromium. In some cases the kits further include instructions for consuming amounts of the soy protein and the chromium effective to inhibit the loss of lean body mass under conditions of caloric restriction. In some instances the kits also include instructions for a calorically restricted diet.

The soy protein and chromium are provided in any form disclosed above, such as in a single combined composition, or separately. The instructions provided with the kit are in a fixed form, such as written or recorded onto an audiocassette, videocassette, compact disc, or digital videodisc. The instructions instruct an individual about amounts of the soy protein and chromium to consume in order to inhibit the loss of lean body mass while dieting. The instructions may also include dietary instructions, as discussed above. In some cases the kits contain a composition of soy protein and chromium, such as a shake drink comprising soy protein and chromium. In other cases the composition is provided in the form of a dry drink powder. In some instances the composition is provided in bulk form, with more than one serving per container. In such cases the instructions instruct an individual to consume a certain amount of the composition per day. In other instances, the composition is provided in containers having single servings. In these cases the instructions instruct an individual to consume a certain number of single servings per day. In both cases an individual following the instructions consumes an amount of soy protein and chromium effective to inhibit the loss of lean body mass under conditions of caloric restriction. The composition may be sold without the instructions for using the product, for example, it is consumed, sold, or administered for weight loss purposes, or for inhibiting loss of lean body mass during periods of caloric restriction.

Some embodiments of the kits further include corosolic acid (and optionally the additional chromium and the other disclosed components of the nutritional supplement) in any form in amounts effective to cause subjects to lose weight, such as part of the disclosed soy protein/chromium composition or separately, as described above. In particular embodiments the kits include the nutritional supplement described above in tablet or capsule form.

Still further embodiments of the kits include the disclosed dietary composition in amounts effective to cause subjects to lose weight. In particular cases the dietary composition is provided in a spray bottle adapted to deliver a dose of about 0.1-0.5 mL of the dietary composition per spray.

The following are merely examples of particular embodiments of the compositions, methods, and kits described herein and are not intended to be limiting in any way.

### EXAMPLE 1

One embodiment of a composition for inhibiting the loss of lean body mass under conditions of caloric restriction is a nutritional shake drink containing chromium polynicotinate and soy protein. In this specific example, the composition also contains healthy fat, healthy, low-glycemic index carbohydrates, and fiber, along with vitamins and minerals as shown in Table 1.

This drink is prepared by adding liquid, such as water or juice to the following powdered formulation and shaking the mixture.

TABLE 1

| | Percent by Weight of Powder |
|---|---|
| Ingredients | (% w/w) |
| Soy Protein | 20-40 |
| Other Proteins | 0-10 |
| Carbohydrates | 40-60 |
| Fats | 5-20 |
| Fiber | 10-20 |
| Chromium | Trace (80-800 μg) in the form of |
| | Chromium Nicotinate |
| Vitamins and Minerals | q.s. |
| | (as much as suffices) |
| Other Ingredients | q.s. |
| Total | 100 |

### EXAMPLE 2

Another embodiment of a composition for inhibiting the loss of lean body mass under conditions of caloric restriction is a premixed, nutritional shake drink containing chromium polynicotinate and soy protein. In this specific example, the composition also contains healthy fat, healthy, low-glycemic index carbohydrates, and fiber, along with vitamins and minerals as shown in Table 2.

This drink is ready for consumption without any mixing.

TABLE 2

| | Percent by Weight of Drink |
|---|---|
| Ingredients | (% w/w) |
| Purified Water | 60-95 |
| Soy Protein | 2-15 |
| Other Proteins | 0-5 |
| Carbohydrates | 2-12 |
| Fats | 0.5-5 |
| Fiber | 5-15 |
| Chromium | Trace (80-800 μg) in the form of |
| | Chromium Nicotinate |
| Vitamins and Minerals | q.s. |
| Other Ingredients | q.s. |
| Total | 100 |

### EXAMPLE 3

This example is one embodiment of a method of using a combination of soy protein and chromium in a method of losing weight under conditions of caloric restriction. The soy protein is consumed in the form of a shake drink while the chromium is consumed in the form of a separate capsule. The formulations for each are shown below in Table 3 and Table 4.

### EXAMPLE 3A

### Drink Formulation

TABLE 3

| | Percent by Weight of Powder |
|---|---|
| Ingredients | (% w/w) |
| Soy Protein | 20-40 |
| Other Proteins | 0-10 |
| Carbohydrates | 20-60 |
| Fats | 5-20 |
| Fiber | 5-20 |
| Vitamins and Minerals | q.s. |
| Other Ingredients | q.s. |
| Total | 100 |

### EXAMPLE 3B

### Formulation for Capsule Product

TABLE 4

| | Percent by Weight of Capsule |
|---|---|
| Ingredients | (% w/w) |
| Maltodextrin | 0-60 |
| Silicon | 0-50 |
| Dioxide | |
| Chromium | Trace (80-360 μg) in the form of |
| | Chromium Nicotinate |
| Other Ingredients | q.s. |
| Total | 100 |

In this specific example, one shake drink is consumed each day as meal replacement or supplement and one chromium capsule is also consumed each day. The shake drink is prepared by adding liquid, such as purified water to the powdered formulation and shaking. An individual also consumes additional calories, but no more than the individual expends in the day.

### EXAMPLES 4 AND 5

Other specific embodiments of compositions for inhibiting the loss of lean body mass under conditions of caloric restriction are shake drinks including chromium nicotinate and soy protein. In these specific examples the compositions also include healthy fat, healthy, low-glycemic index carbohydrates, and fiber, along with vitamins and minerals as shown in Table 5. This example also contains a method of using these shake drinks can by consuming the shakes as meal replacements or dietary supplements. In this particular example, one shake drink is consumed each day.

These shake drinks are prepared by adding liquid, such as water or juice to the following powdered formulation and then shaking the mixture. In these specific examples about 47 to about 55 grams of the powder are combined with about 8 ounces of water.

TABLE 5

| | Example 4 | | | Example 5 | | |
|---|---|---|---|---|---|---|
| | Percent by | | | Percent by | | |
| | Weight of | | | Weight of | | |
| | Powder | Weight/ | | Powder | Weight/ | |
| Ingredients | (% w/w) | Serving | | (% w/w) | Serving | |
| Soy Protein | 29.40 | 13.91 | g | 27.71 | 13.91 | g |
| Isolate | | | | | | |
| Fructose | 23.43 | 11.08 | g | 28.06 | 14.08 | g |
| High Oleic | 12.68 | 6.00 | g | 11.76 | 5.90 | g |
| Sunflower | | | | | | |
| Oil Powder | | | | | | |
| Acacia Gum | 15.85 | 7.50 | g | 14.94 | 7.50 | g |
| Canola Oil | 2.56 | 1.07 | g | 2.20 | 1.10 | g |
| Inulin | 2.11 | 1.00 | g | 1.99 | 1.00 | g |
| Milk Protein | 1.90 | 0.90 | g | 1.79 | 0.90 | g |
| Isolate | | | | | | |
| Dicalcium | 2.10 | 0.99 | g | 1.98 | 0.99 | g |
| Phosphate | | | | | | |
| Silicon | 1.69 | 0.80 | g | 1.04 | 0.52 | g |
| Dioxide | | | | | | |
| Sodium | 1.23 | 0.58 | g | 1.16 | 0.58 | g |
| Citrate | | | | | | |
| Potassium | 1.20 | 0.57 | g | 1.13 | 0.57 | g |
| Chloride | | | | | | |
| Soy Lecithin | 1.06 | 0.50 | g | 1.10 | 0.55 | g |
| Whey Protein | 0.95 | 0.45 | g | 0.90 | 0.45 | g |
| Isolate | | | | | | |
| Guar Gum | 1.90 | 0.90 | g | 1.79 | 0.90 | g |
| Flavoring | 0.79 | 0.37 | g | 1.10 | 0.55 | g |
| Vitamin | 0.45 | 0.21 | g | 0.42 | 0.21 | g |
| Premix | | | | | | |
| Mineral | 0.37 | 0.18 | g | 0.35 | 0.18 | g |
| Premix | | | | | | |
| Chromium | 0.003 | 1.4 | mg | 0.003 | 1.4 | mg |
| Nicotinate | | (provides | | | (provides | |
| | | 180 | μg | | 180 | μg |
| | | chromium) | | | chromium) | |
| Ferrous | 0.025 | 11.9 | mg | 0.024 | 11.9 | mg |
| Fumarate | | | | | | |
| Sucralose | 0.0085 | 4.0 | mg | | | |
| Mixed | 0.002 | 0.9 | mg | 0.002 | 0.9 | mg |
| Tocopherol | | | | | | |
| Concentrate | | | | | | |
| TOTAL | 100.00 | 47.31 | g | 100 | 50.19 | g |

### EXAMPLE 6

One embodiment of the disclosed nutritional supplement that is combined with the disclosed soy protein/chromium combination includes capsules comprising about 16 mg of banaba leaf extract standardized to contain about 1% corosolic acid and about 200 μg chromium, such as chromium polynicotinate, as well as about 100 mg magnesium, about 2.5 μg zinc, about 250 mg taurine, about 50 μg vanadium, and about 5 mg alpha lipoic acid, such that two capsules contain an effective daily amount of the nutritional supplement, for example 32 mg of banaba leaf extract standardized to contain about 1% corosolic acid and about 400 μg chromium, as well as about 200 mg magnesium, about 5 μg zinc, about 500 mg taurine, about 100 μg vanadium, and about 10 mg alpha lipoic acid. The capsules include a shell of hard gelatin comprising gelatin and water. The nutritional supplement is mixed with maltodextrin for encapsulation.

### EXAMPLE 7

One embodiment of the dietary composition that is combined with the disclosed soy protein/chromium combination and the disclosed nutritional supplement includes equal amounts of dilutions of about 3X Fucus vesiculosus, about 3X Gambogia garcinia, about 30C Apis mellifica, about 4X Badiaga, about 30X Calcarea carbonica, about 3X Passiflora incarnata, about 12X Baryta carbonica, about 12X Calcarea fluorica, about 6X Lycopodium clavatum, about 6X Berberis vulgaris, about 3X Leptandra virginica, about 6X Thuja occidentalis, about 3X Galium aparine, about 30C Urtica urens, about 200C Histaminum muriaticum, and about 30C Sabadilla, which are mixed with about 20% by volume of alcohol and provided in a spray bottle adapted to spray about 0.1-0.5 mL of the dietary composition per spray.

### EXAMPLE 8

This example demonstrates that overweight subjects under conditions of caloric restriction that consume each day an embodiment of the disclosed soy/chromium composition including at least about 12 g of soy protein and at least about 180 μg of chromium lose weight while inhibiting the loss of lean body mass. The subjects in this study consuming the soy protein and chromium composition lost less lean body mass in total and as a percentage of total weight lost than subjects on the same calorie diet that did not consume the composition.

This study was conducted using 72 subjects, of which 54 were women and 18 were men. Subjects in this study were determined to be clinically overweight or obese as measured by their body mass index. Subjects in this study comprised two groups, each having 27 women and 9 men. One group (Diet and Exercise Group—with Shake) consumed the soy/chromium composition of Example 4, containing 200 kcal, about 12-14 grams of soy protein and about 180 μg of chromium for one meal a day. These subjects also followed a self-policed 1,000 kcal/day diet for 2 additional meals per day for a total intake of 1,200 kcal per day. This group also was allowed to exercise at will. A second group (Diet and Exercise—without Shake) consumed 1,200 kcal/day and was allowed to exercise at will, but did not consume a soy protein and chromium composition.

The loss of fat mass and lean body mass for these subjects was obtained from DEXA (dual energy x-ray absorptiometry) measurements. This technique accurately measures the amount of fat and bone present in the human body. By calculation, the amount of lean body mass can then be obtained.

A baseline weight, and fat and lean body mass was established for each subject, and the subjects were again tested after 8 weeks. The amounts of weight, and fat and lean body mass lost on average by each subject in each group at 8 weeks is shown in the following table.

TABLE 6

| | Diet and Exercise | Diet and Exercise |
|---|---|---|
| | Group - | Group - |
| Parameter Measured | with Shake | without Shake |
| Weight Lost (kg) | 4.3 | 5.1 |
| Fat Lost (kg) | 2.6 | 2.8 |
| Lean Body Mass Lost | 1.4 | 2.1 |
| (kg) | | |
| Lean Body Mass Lost | 32.6% | 41.2% |
| (% of Total Weight | | |
| Lost) | | |
| Ratio of Lean Body Mass | 0.53 | 0.75 |
| Lost to Fat Mass Lost | | |

As can be seen from these results, both groups lost weight. For the diet and exercise group that did not consume the soy and chromium composition, 41.2% of the weight lost was lean body mass. Also, the ratio of lean body mass lost to fat mass lost was 0.75. Better results were observed in the diet and exercise group that consumed the soy and chromium composition. In this group only 32.6% of the weight lost was lean body mass. Also, the ratio of lean body mass lost to fat mass lost was 0.53. Further, the total amount of lean body mass lost by the group consuming the soy protein and chromium composition was significantly less than the amount lost by the group that did not consume the composition. The group that did not consume the shake lost on average 2.1 kg of lean body mass, while the group that consumed the shake lost only 1.4 kg of lean body mass.

Under the regimen of caloric restriction established in this study, the subjects consuming the soy protein and chromium composition should have lost an equal amount of lean body mass as the diet only group. However, consuming the composition resulted in an inhibition of the loss of lean body mass relative to the lean body mass lost in subjects not consuming the composition.

As an added benefit, when asked about the sensation of satiety (feeling of fullness) following the ingestion of the composition, test subjects responded favorably. These same subjects also responded positively when questioned about the ability of this composition to reduce cravings for food while using this product. Additionally, this composition has been shown to help retain normal blood glucose levels while minimizing the spike in insulin levels created by other weight loss drinks.

### EXAMPLE 9

This example demonstrates the effectiveness of the disclosed soy protein/chromium composition, the disclosed nutritional supplement, and the disclosed dietary supplement in causing weight loss and inhibition of loss of lean body mass.

A study was conducted using 128 subjects, of which 87 were women and 41 were men. Subjects in this study were determined to be clinically overweight or obese as measured by their body mass index. Subjects in this study included three groups, each having about 43 subjects at baseline and between 36 and 38 subjects at eight weeks (some subjects could no longer comply). One group (Diet and Exercise Group) was given general guidelines regarding diet and exercise, but was not required to adhere to any particular exercise regimen or calorically restricted diet. A second group (Supplement+Spray (SS)) consumed the nutritional supplement of Example 6 and the dietary composition of Example 7 and received the same general guidelines regarding diet and exercise, but was not required to adhere to any particular exercise regimen or calorically restricted diet. This group took two tablets of the Supplement immediately before the largest meal of the day, and took one dose of the Spray (2 interval sprays of the spray from a spray bottle adapted to spray about 0.2 mL per spray) about 15-30 minutes before lunch and dinner. These subjects used the spray on alternating intervals of three weeks using the spray (“on”) and one week not using the spray (“off”) during the study. A third group (Supplement+Spray+Shake (SSS)) consumed the Shake composition of Example 4, containing 200 kcal, about 12-14 grams of soy protein and about 180 μg of chromium for breakfast each day and used the Supplement and Spray as indicated for the SS group. These subjects also received the same general guidelines regarding diet and exercise, but were not required to adhere to any particular exercise regimen or calorically restricted diet.

The loss of fat mass and lean body mass for these subjects was obtained from DEXA (dual energy x-ray absorptiometry) measurements. This technique accurately measures the amount of fat and bone present in the human body. By calculation, the amount of lean body mass can then be obtained.

A baseline weight, and fat and lean body mass was established for each subject, and the subjects were again tested after 8 weeks. The amounts of weight, and fat and lean body mass lost on average by each subject in each group at 8 weeks are shown in the following table.

TABLE 7

| | Diet and Exercise | | |
|---|---|---|---|
| Parameter Measured | Group | SS Group | SSS Group |
| Weight Lost (kg) | 5.1 | 5.3 | 4.2 |
| Fat Lost (kg) | 1.6 | 1.4 | 1.5 |
| Lean Body Mass Lost | 2.1 | 2.1 | 1.4 |
| (kg) | | | |
| Lean Body Mass Lost | 41% | 40% | 33.3% |
| (% of Total Weight | | | |
| Lost) | | | |
| Ratio of Lean Body | 1.31 | 1.5 | 0.93 |
| Mass Lost to Fat | | | |
| Mass Lost | | | |

As can be seen from these results, all groups lost weight. For the diet and exercise group that did not use either the SS or SSS, 41% of the weight lost was lean body mass. Also, the ratio of lean body mass lost to fat mass lost was 1.31 (more lean body mass was lost than fat). Not much better results were observed in the SS group that used Supplement and the Spray.

However, subjects using the Shake, Supplement, and Spray lost only 33.3% of their weight as lean body mass with a ratio of lean body mass lost to fat lost of somewhat less than 1 (more of the weight lost was fat than lean body mass).

As all groups were free to eat and exercise at will outside of the requirements to use the study products, the SSS subjects should have lost an equal amount of lean body mass as the other two groups. However, using the combination of products including the Shake, Supplement and Spray resulted in an inhibition of the loss of lean body mass relative to the lean body mass lost in subjects not consuming these products, even without conscious caloric restriction.

### EXAMPLE 10

This example demonstrates the effectiveness of the disclosed soy protein/chromium composition, the disclosed nutritional supplement, and the disclosed dietary composition in causing weight loss in subjects without conscious caloric restriction, such as specific instructions to restrict caloric intake.

A study was conducted using 123 subjects, of which 61 were women and 62 were men. Subjects in this study were determined to be clinically overweight or obese as measured by their body mass index. Subjects in this study were divided into three groups, each having about 41 subjects at baseline and between 35 and 39 subjects at twelve weeks (some subjects could no longer comply). One group (Supplement+Spray+Shake (SSS)) consumed the Shake composition of Example 4, containing 200 kcal, about 12-14 grams of soy protein and about 180 μg of chromium with breakfast each day (subjects were instructed to consume the shake before consuming any food at breakfast and instructed that a regular breakfast meal could be consumed thereafter) and used the nutritional supplement of Example 7 and the dietary composition of Example 8. This group took two tablets of the Supplement immediately before the subjects' largest meal of the day, and took one dose of the Spray about 15-30 minutes before lunch and dinner. These subjects took the spray on a three weeks on/one week off basis during the study. These subjects engaged in exercise by walking one mile five days a week. A second group (Placebo/Sedentary (PS)) used placebo Supplements (magnesium oxide and maltodextrin) and Spray (purified water and alcohol) as indicated for the SSS group and was instructed to refrain from participation in any exercise program. A third group (Placebo/Exercise (PE)) used placebo Supplements (magnesium oxide and maltodextrin) and Spray (purified water and alcohol) as indicated for the SSS group and followed the same exercise regimen as the SSS group. None of the groups were instructed concerning caloric restriction or asked to follow any certain diet other than using the study products.

The loss of fat mass for these subjects was obtained from DEXA (dual energy x-ray absorptiometry) measurements. This technique accurately measures the amount of fat and bone present in the human body.

A baseline weight, and fat mass was established for each subject, and the subjects were again tested after 12 weeks. The amounts of weight, and fat lost on average by each subject in each group at 12 weeks are shown in the following table. Also shown are the average amounts of non-fat mass lost calculated from the amounts of fat lost and weight loss, which represents both lean body mass lost as well as loss of any other non-fat body mass, such as water and bone. The amount of lean-body mass lost likely was less than the non-fat mass lost.

TABLE 8

| Parameter Measured | SSS Group | PS Group | PE Group |
|---|---|---|---|
| Weight Lost (kg) | .91 | (.89) | (.68) |
| Fat Lost (kg) | .46 | (.73) | (.37) |
| Non-fat | .45 | (.16) | (.31) |
| Mass Lost (kg) | | | |
| Non-fat Mass Lost | 49.5% | NA | NA |
| (% of Total | | | |
| Weight Loss) | | | |
| Ratio of Non-fat Mass | .98 | NA | NA |
| Lost to Fat Mass Lost | | | |

As can be seen from these results, only the SSS group lost weight. The other two groups gained weight. As expected, the PE group gained less weight than the PS group. However, as both the SSS and PE groups were free to eat at will and were required to participate in identical exercise regimens the SSS subjects should have gained an equal amount of weight as the PE group. However, consuming the combination of products including the Shake, Supplement, and Spray resulted in a loss of weight even without conscious caloric restriction.

### EXAMPLE 11

This example demonstrates the effectiveness of the disclosed soy/chromium composition, the disclosed nutritional supplement, and the disclosed dietary composition in causing weight loss in subjects with Metabolic Syndrome.

This example relies on the same study as described in Example 10. However, only subjects in the SSS group were selected for a determination of the effect of the SSS combination on subjects with Metabolic Syndrome. In the SSS group, 15 subjects had Metabolic Syndrome and 20 subjects did not (of the subjects remaining in the study at the 12^{th} week). The following table summarizes the results for weight change and fat loss for subjects in the SSS group with Metabolic Syndrome and without.

TABLE 9

| | Metabolic | No Metabolic |
|---|---|---|
| Parameter Measured | Syndrome | Syndrome |
| Weight Lost (kg) | 1.34 | .82 |
| Fat Lost (kg) | .71 | .27 |
| Non-fat Mass Lost (kg) | .63 | .55 |
| Non-fat Mass Lost | 47% | 67% |
| (% of Total Weight Lost) | | |
| Ratio of Non-fat Mass Lost | .89 | 2.03 |
| to Fat Mass Lost | | |

As can be seen from the results, subjects having Metabolic Syndrome lost more weight than subjects without Metabolic Syndrome and lost a greater portion of the weight lost as fat.

The above-described examples merely disclose particular, specific embodiments of the disclosed compositions, methods, and kits. They are not intended to be limiting in any way. Moreover, although these embodiments have been described in detail, those of ordinary skill in the art will understand that variations may be made thereto without departing from the spirit of the invention or scope of the appended claims.

We claim:

1. A composition effective for inhibiting the loss of lean body mass in a subject under conditions of caloric restriction comprising:
about 8 grams or more of soy protein, and
about 100 micrograms or more of chromium, wherein the chromium is in the form of a biologically acceptable salt or chelate of the chromium.

2. The composition of claim 1, wherein the composition comprises about 10 grams or more of the soy protein and about 120 micrograms or more of the chromium.

3. The composition of claim 2, wherein the composition comprises about 12 grams or more of the soy protein and about 180 micrograms or more of the chromium.

4. The composition of claim 3, wherein the composition comprises about 12 grams to about 14 grams of the soy protein.

5. The composition of claim 3, wherein the biologically acceptable salt or chelate of the chromium comprises chromium nicotinate or chromium picolinate or compositions thereof.

6. The composition of claim 5, wherein the composition comprises, in percentage by weight of the total weight of the composition, about 10% to about 50% of the soy protein and about 0.0005% to about 0.10% of the chromium, wherein the biologically acceptable salt or chelate of the chromium comprises chromium nicotinate, and the composition further comprises, in percentage by weight of the total weight of the composition,

| | |
|---|---|
| Fructose | about 20% to about 30% |
| High Oleic Sunflower Oil Powder | about 8% to about 18% |
| Acacia Gum | about 10% to about 15% |
| Canola Oil | about 1% to about 5% |
| Inulin | about 1% to about 5% |
| Milk Protein Isolate | about 0.5% to about 5% |
| Dicalcium Phosphate | about 0.5% to about 3% |
| Silicon Dioxide | about 1% to about 2% |
| Sodium Citrate | about 0.5% to about 3% |
| Potassium Chloride | about 0.5% to about 3% |
| Soy Lecithin | about 0.5% to about 3% |
| Whey Protein Isolate | about 0.05% to about 2% |
| Guar Gum | about 0.1% to about 2% |
| Flavoring | about 1% to about 2% |
| Vitamin Premix | about 0.05% to about 2% |
| Mineral Premix | about 0.05% to about 2% |
| Ferrous Fumarate | about 0.005% to about 0.05% |
| Sucralose | about 0.001% to about 0.10% |
| Mixed Tocopherol Concentrate | about 0.0005% to about 0.005% |

7. The composition of claim 6, wherein the composition comprises, in percentage by weight of the total weight of the composition,

| | |
|---|---|
| Soy Protein Isolate | about 27.5-29.5% |
| Fructose | about 23-28% |
| High Oleic Sunflower Oil Powder | about 11.5-12.75% |
| Acacia Gum | about 14.5-16% |
| Canola Oil | about 2-2.75% |
| Inulin | about 1.75-2.25% |
| Milk Protein Isolate | about 1.75-2% |
| Dicalcium Phosphate | about 1.75-2.25% |
| Silicon Dioxide | about 1-1.75% |
| Sodium Citrate | about 1-1.25% |
| Potassium Chloride | about 1-1.25% |
| Soy Lecithin | about 1-1.10% |
| Whey Protein Isolate | about 0.9-1% |
| Guar Gum | about 1.75-2% |
| Flavoring | about 0.75-1.25% |
| Vitamin Premix | about 0.4-0.45% |
| Mineral Premix | about 0.3-0.4% |
| Chromium Nicotinate | about 0.002-0.004% |
| Ferrous Fumarate | about 0.020-0.030% |
| Mixed Tocopherol Concentrate | about 0.001-0.003% |

8. The composition of claim 7, wherein the composition further comprises about 0.0080-0.0090% sucralose, and comprises,

| | |
|---|---|
| Soy Protein Isolate | about 13.91 grams |
| Fructose | about 11.08 grams |
| High Oleic Sunflower Oil Powder | about 6.00 grams |
| Acacia Gum | about 7.50 grams |
| Canola Oil | about 1.07 grams |
| Inulin | about 1.00 grams |
| Milk Protein Isolate | about 0.90 grams |
| Dicalcium Phosphate | about 0.99 grams |
| Silicon Dioxide | about 0.80 grams |
| Sodium Citrate | about 0.58 grams |
| Potassium Chloride | about 0.57 grams |
| Soy Lecithin | about 0.50 grams |
| Whey Protein Isolate | about 0.45 grams |
| Guar Gum | about 0.90 grams |
| Flavoring | about 0.37 grams |
| Vitamin Premix | about 0.21 grams |
| Mineral Premix | about 0.18 grams |
| Chromium Nicotinate | about 1.4 milligrams |
| | (comprising about |
| | 180 micrograms chromium) |
| Ferrous Fumarate | about 11.9 milligrams |
| Sucralose | about 4.0 milligrams |
| Mixed Tocopherol Concentrate | about 0.9 milligrams |

9. The composition of claim 7, wherein the composition comprises,

| | |
|---|---|
| Soy Protein Isolate | about 13.91 grams |
| Fructose | about 14.08 grams |
| High Oleic Sunflower Oil Powder | about 5.90 grams |
| Acacia Gum | about 7.50 grams |
| Canola Oil | about 1.10 grams |
| Inulin | about 1.00 grams |
| Milk Protein Isolate | about 0.90 grams |
| Dicalcium Phosphate | about 0.99 grams |
| Silicon Dioxide | about 0.52 grams |
| Sodium Citrate | about 0.58 grams |
| Potassium Chloride | about 0.57 grams |
| Soy Lecithin | about 0.55 grams |
| Whey Protein Isolate | about 0.45 grams |
| Guar Gum | about 0.90 grams |
| Flavoring | about 0.55 grams |
| Vitamin Premix | about 0.21 grams |
| Mineral Premix | about 0.18 grams |
| Chromium Nicotinate | about 1.4 milligrams |
| | (comprising about |
| | 180 μg chromium) |
| Ferrous Fumarate | about 11.9 milligrams |
| Mixed Tocopherol Concentrate | about 0.9 milligrams |

10. The composition of claim 5, wherein the composition provides about 100 to about 400 calories.

11. The composition of claim 10, wherein the composition provides about 200 calories.

12. The composition of claim 5, further comprising an amount of corosolic acid effective to inhibit the loss of lean body mass in the subject under caloric restriction.

13. The composition of claim 12, wherein the composition comprises about 0.5-1 mg of corosolic acid.

14. The composition of claim 13, wherein the corosolic acid comprises banaba leaf extract.

15. The composition of claim 14, wherein the composition comprises about 5-100 mg of banaba leaf extract.

16. The composition of claim 15, wherein the composition comprises about 25-35 mg of banaba leaf extract.

17. The composition of claim 13, further comprising an additional 100-600 μg of chromium in the form of a biologically acceptable salt or chelate of the additional chromium.

18. The composition of claim 17, further comprising magnesium, zinc, taurine, vanadium, and alpha lipoic acid.

19. A method for losing weight while inhibiting a loss of lean body mass comprising:
following a calorically restricted diet, and
consuming about 8 grams or more of soy protein per day and about 100 micrograms or more of chromium per day, wherein the chromium is in the form of a biologically acceptable salt or chelate of the chromium, and wherein the amounts of the soy protein and the chromium are effective to inhibit the loss of lean body mass while following the calorically restricted diet.

20. The method of claim 19, comprising consuming about 10 grams or more of the soy protein and about 120 micrograms or more of the chromium per day.

21. The method of claim 20, comprising consuming about 12 grams or more of the soy protein and about 180 micrograms or more of the chromium per day.

22. The method of claim 21, comprising consuming about 12 grams to about 14 grams of the soy protein per day.

23. The method of claim 19, wherein consuming the soy protein comprises consuming the soy protein in a shake drink, and wherein consuming the chromium comprises consuming the chromium separately in a pharmaceutically acceptable dosage form.

24. The method of claim 19, wherein the soy protein and the chromium are consumed as a soy protein/chromium composition.

25. The method of claim 24, wherein the soy protein/chromium composition provides about 100 to about 400 calories.

26. The method of claim 25, wherein the soy protein/chromium composition provides about 200 calories.

27. The method of claim 19, comprising following the calorically restricted diet and consuming the soy protein and the chromium for a period of about five days or longer.

28. The method of claim 27, comprising following the calorically restricted diet and consuming the soy protein and the chromium for a period of from about 8 weeks to about 12 weeks.

29. The method of claim 28, wherein losing weight includes losing fat mass, and wherein the ratio of lean body mass lost to fat mass lost is less than about 0.6.

30. The method of claim 19, wherein following the calorically restricted diet comprises consuming 2,000 calories or less per day.

31. The method of claim 30, wherein following the calorically restricted diet comprises consuming 1,400 calories or less per day.

32. The method of claim 31, wherein following the calorically restricted diet comprises consuming 1,200 calories or less per day.

33. The method of claim 19, wherein the biologically acceptable salt or chelate of the chromium comprises chromium nicotinate or chromium picolinate or compositions thereof.

34. The method of claim 24, wherein the soy protein/chromium composition comprises, in percentage by weight of the total weight of the soy protein/chromium composition, about 10% to about 50% of the soy protein and about 0.0005% to about 0.10% of the chromium, wherein the biologically acceptable salt or chelate of the chromium comprises chromium nicotinate, and further comprises, in percentage by weight of the total weight of the soy protein/chromium composition,

| | |
|---|---|
| Fructose | about 20% to about 30% |
| High Oleic Sunflower Oil Powder | about 8% to about 18% |
| Acacia Gum | about 10% to about 15% |
| Canola Oil | about 1% to about 5% |
| Inulin | about 1% to about 5% |
| Milk Protein Isolate | about 0.5% to about 5% |
| Dicalcium Phosphate | about 0.5% to about 3% |
| Silicon Dioxide | about 1% to about 2% |
| Sodium Citrate | about 0.5% to about 3% |
| Potassium Chloride | about 0.5% to about 3% |
| Soy Lecithin | about 0.5% to about 3% |
| Whey Protein Isolate | about 0.05% to about 2% |
| Guar Gum | about 0.1% to about 2% |
| Flavoring | about 1% to about 2% |
| Vitamin Premix | about 0.05% to about 2% |
| Mineral Premix | about 0.05% to about 2% |
| Ferrous Fumarate | about 0.005% to about 0.05% |
| Sucralose | about 0.001% to about 0.10% |
| Mixed Tocopherol Concentrate | about 0.0005% to about 0.005% |

35. The method of claim 34, wherein the soy protein/chromium composition comprises, in percentage by weight of the total weight of the soy protein/chromium composition,

| | |
|---|---|
| Soy Protein Isolate | about 27.5-29.5% |
| Fructose | about 23-28% |
| High Oleic Sunflower Oil Powder | about 11.5-12.75% |
| Acacia Gum | about 14.5-16% |
| Canola Oil | about 2-2.75% |
| Inulin | about 1.75-2.25% |
| Milk Protein Isolate | about 1.75-2% |
| Dicalcium Phosphate | about 1.75-2.25% |
| Silicon Dioxide | about 1-1.75% |
| Sodium Citrate | about 1-1.25% |
| Potassium Chloride | about 1-1.25% |
| Soy Lecithin | about 1-1.10% |
| Whey Protein Isolate | about 0.9-1% |
| Guar Gum | about 1.75-2% |
| Flavoring | about 0.75-1.25% |
| Vitamin Premix | about 0.4-0.45% |
| Mineral Premix | about 0.3-0.4% |
| Chromium Nicotinate | about 0.002-0.004% |
| Ferrous Fumarate | about 0.020-0.030% |
| Mixed Tocopherol Concentrate | about 0.001-0.003% |

36. The method of claim 35, wherein the soy protein/chromium composition further comprises about 0.0080-0.0090% sucralose, and comprises,

| | |
|---|---|
| Soy Protein Isolate | about 13.91 grams |
| Fructose | about 11.08 grams |
| High Oleic Sunflower Oil Powder | about 6.00 grams |
| Acacia Gum | about 7.50 grams |
| Canola Oil | about 1.07 grams |
| Inulin | about 1.00 grams |
| Milk Protein Isolate | about 0.90 grams |
| Dicalcium Phosphate | about 0.99 grams |
| Silicon Dioxide | about 0.80 grams |
| Sodium Citrate | about 0.58 grams |
| Potassium Chloride | about 0.57 grams |
| Soy Lecithin | about 0.50 grams |
| Whey Protein Isolate | about 0.45 grams |
| Guar Gum | about 0.90 grams |
| Flavoring | about 0.37 grams |
| Vitamin Premix | about 0.21 grams |
| Mineral Premix | about 0.18 grams |
| Chromium Nicotinate | about 1.4 milligrams |
| | (comprising about |
| | 180 μg chromium) |
| Ferrous Fumarate | about 11.9 milligrams |
| Sucralose | about 4.0 milligrams |
| Mixed Tocopherol Concentrate | about 0.9 milligrams |
| TOTAL | about 47.31 grams |

37. The method of claim 35, wherein the soy protein/chromium composition comprises,

| | |
|---|---|
| Soy Protein Isolate | about 13.91 grams |
| Fructose | about 14.08 grams |
| High Oleic Sunflower Oil Powder | about 5.90 grams |
| Acacia Gum | about 7.50 grains |
| Canola Oil | about 1.10 grams |
| Inulin | about 1.00 grams |
| Milk Protein Isolate | about 0.90 grams |
| Dicalcium Phosphate | about 0.99 grams |
| Silicon Dioxide | about 0.52 grams |
| Sodium Citrate | about 0.58 grams |
| Potassium Chloride | about 0.57 grams |
| Soy Lecithin | about 0.55 grams |
| Whey Protein Isolate | about 0.45 grams |
| Guar Gum | about 0.90 grams |
| Flavoring | about 0.55 grams |
| Vitamin Premix | about 0.21 grams |
| Mineral Premix | about 0.18 grams |
| Chromium Nicotinate | about 1.4 milligrams |
| | (comprising about |
| | 180 μg chromium) |
| Ferrous Fumarate | about 11.9 milligrams |
| Mixed Tocopherol Concentrate | about 0.9 milligrams |
| TOTAL | about 50.19 grams |

38. The method of claim 19, further comprising consuming corosolic acid in an amount effective to inhibit the loss of the subject's lean body mass while losing weight under conditions of caloric restriction.

39. The method of claim 38, wherein consuming the corosolic acid comprises consuming about 0.5-1 mg of corosolic acid.

40. The method of claim 39, wherein the corosolic acid comprises banaba leaf extract.

41. The method of claim 40, wherein consuming the corosolic acid comprises consuming about 5-100 mg of the banaba leaf extract.

42. The method of claim 41, wherein consuming the corosolic acid comprises consuming about 25-35 mg of the banaba leaf extract.

43. The method of claim 39, further comprising consuming an additional 100-600 μg of chromium in the form of a biologically acceptable salt or chelate of the additional chromium.

44. The method of claim 43, further comprising consuming magnesium, zinc, taurine, vanadium, and alpha lipoic acid.

45. The method of claim 43, wherein the corosolic acid and additional chromium are consumed as a nutritional supplement.

46. The method of claim 45, wherein the nutritional supplement is consumed prior to one or more meals a day.

47. The method of claim 43, further comprising consuming a dietary composition comprising Fucus vesiculosus, Gambogia garcinia, Apis mellifica, Badiaga, Calcarea carbonica, Passiflora incarnata, Baryta carbonica, Calcarea fluorica, Lycopodium clavatum, Berberis vulgaris, Leptandra virginica, Thuja occidentalis, Galium aparine, Urtica urens, Histaminum muriaticum, and Sabadilla in an amount effective to inhibit the loss of the subject's lean body mass while losing weight under conditions of caloric restriction.

48. The method of claim 47, wherein the dietary composition is consumed prior to one or more meals a day.

49. The method of claim 47, wherein the calorically restricted diet is not consciously followed.

50. A method for treatment of an overweight subject desiring to lose weight comprising:
selecting a subject that is overweight and desires to lose weight, and
administering to the subject soy protein and chromium, wherein the chromium is in the form of a biologically acceptable salt or chelate of the chromium, in amounts effective to inhibit the loss of the subject's lean body mass while losing weight under conditions of caloric restriction.

51. The method of claim 50, wherein selecting a subject that is overweight comprises selecting a subject having a body mass index of at least about 25.

52. The method of claim 51, wherein selecting a subject that is overweight comprises selecting a subject having a body mass index of at least about 30.

53. The method of claim 51, wherein selecting a subject that is overweight further comprises selecting a subject with Metabolic Syndrome.

54. The method of claim 50, further comprising instructing the subject about a calorically restricted diet.

55. The method of claim 54, wherein instructing the subject about a calorically restricted diet comprises instructing the subject to consume 2,000 calories or less per day.

56. The method of claim 55, wherein instructing the subject about a calorically restricted diet comprises instructing the subject to consume 1,400 calories or less per day.

57. The method of claim 56, wherein instructing the subject about a calorically restricted diet comprises instructing the subject to consume 1,200 calories or less per day.

58. The method of claim 50, wherein losing weight includes losing fat mass, and wherein administering the soy protein and the chromium is effective to result in a ratio of lean body mass lost to fat mass lost of less than about 0.6.

59. The method of claim 50, comprising administering about 10 grams or more of the soy protein and about 120 micrograms or more of the chromium per day.

60. The method of claim 59, comprising administering about 12 grams or more of the soy protein and about 180 micrograms or more of the chromium per day.

61. The method of claim 60, comprising administering about 12 grams to about 14 grams of the soy protein per day.

62. The method of claim 59, wherein the soy protein comprises administering the soy protein in a shake drink, and wherein administering the chromium comprises administering the chromium separately in a pharmaceutically acceptable chromium dosage form.

63. The method of claim 59, wherein the soy protein and the chromium are administered as a soy protein/chromium composition.

64. The method of claim 63, wherein the soy protein/chromium composition provides about 100 to about 400 calories.

65. The method of claim 50, comprising administering the soy protein and the chromium for a period of at least five days.

66. The method of claim 65, comprising administering the soy protein and the chromium for a period of from about 8 weeks to about 12 weeks.

67. The method of claim 50, wherein the biologically acceptable salt or chelate of the chromium comprises chromium nicotinate or chromium picolinate or combinations thereof.

68. The method of claim 50, further comprising administering corosolic acid to the subject in an amount effective to inhibit the loss of the subject's lean body mass while losing weight under conditions of caloric restriction.

69. The method of claim 68, wherein administering the corosolic acid comprises administering about 0.5-1 mg of corosolic acid.

70. The method of claim 69, further comprising administering to the subject an additional 100-600 μg of chromium in the form of a biologically acceptable salt or chelate of the additional chromium effective to inhibit the loss of the subject's lean body mass while losing weight under conditions of caloric restriction.

71. The method of claim 70, wherein the corosolic acid and the additional chromium are administered to the subject as a separate nutritional supplement.

72. The method of claim 71, wherein the nutritional supplement is administered prior to one or more meals a day.

73. The method of claim 71, further comprising administering to the subject a dietary composition comprising Fucus vesiculosus, Gambogia garcinia, Apis mellifica, Badiaga, Calcarea carbonica, Passiflora incarnata, Baryta carbonica, Calcarea fluorica, Lycopodium clavatum, Berberis vulgaris, Leptandra virginica, Thuja occidentalis, Galium aparine, Urtica urens, Histaminum muriaticum, and Sabadilla in an amount effective to inhibit the loss of the subject's lean body mass while losing weight under conditions of caloric restriction.

74. The method of claim 73, wherein the dietary composition is administered prior to one or more meals a day.

75. A kit for inhibiting loss of a subject's lean body mass under conditions of caloric restriction comprising:
soy protein;
chromium in the form of biologically acceptable salt or chelate of the chromium; and
instructions for consuming amounts of the soy protein and the chromium effective to inhibit the loss of the subject's lean muscle mass under conditions of caloric restriction.

76. The kit of claim 75, wherein the biologically acceptable salt or chelate of the chromium comprises chromium nicotinate or chromium picolinate or compositions thereof.

77. The kit of claim 76, wherein the instructions instruct the subject to consume about 12 grams or more of the soy protein per day.

78. The kit of claim 77, wherein the instructions instruct the subject to consume about 120 micrograms or more of the chromium per day.

79. The kit of claim 78, wherein the instructions instruct the subject to consume about 180 micrograms or more of the chromium per day.

80. The kit of claim 79, wherein the instruction further instruct the subject to consume about 1800 kcal/day or less.

81. The kit of claim 78, wherein the soy protein and chromium are provided in one or more doses of 12 grams or more of the soy protein and 120 grams or more of the chromium and the instructions instruct the subject to consume one or more of the doses of the soy protein and the chromium per day.

82. The kit of claim 81, wherein each dose of the soy protein and the chromium is provided in a soy protein/chromium composition comprising the soy protein and the chromium.

83. The kit of claim 82, further comprising corosolic acid and instructions for consuming the corosolic acid in an amount effective to inhibit the loss of the subject's lean muscle mass under conditions of caloric restriction.

84. The kit of claim 83, wherein the corosolic acid is provided in one or more doses of a nutritional supplement comprising about 0.5-1 mg of the corosolic acid.

85. The kit of claim 84, wherein the nutritional supplement further comprises 100-600 μg of additional chromium in the form of a biologically acceptable salt or chelate of the additional chromium effective to inhibit the loss of the subject's lean muscle mass under conditions of caloric restriction.

86. The method of claim 85, wherein the instructions instruct the subject to consume the nutritional supplement prior to one or more meals a day.

87. The kit of claim 83, further comprising a dietary composition comprising Fucus vesiculosus, Gambogia garcinia, Apis mellifica, Badiaga, Calcarea carbonica, Passiflora incarnata, Baryta carbonica, Calcarea fluorica, Lycopodium clavatum, Berberis vulgaris, Leptandra virginica, Thuja occidentalis, Galium aparine, Urtica urens, Histaminum muriaticum, and Sabadilla and instructions for consuming the dietary supplement acid in an amount effective to inhibit the loss of the subject's lean muscle mass under conditions of caloric restriction.

88. The kit of claim 87, wherein the instructions instruct the subject to consume the dietary composition prior to one or more meals a day.
